(19) United States
(12) Patent Application Publication
(10) Pub. No.: US 2012/0258993 A1
(43) Pub. Date: Oct. 11, 2012

(54) NON-NATURAL MACROCYCLIC AMIDE HDAC6 INHIBITOR COMPOUNDS AND THEIR USES AS THERAPEUTIC AGENTS

(75) Inventors: Giuseppe Giannini, Pomezia (RM) (IT); Walter Cabri, Rozzano (MI) (IT); Stephan Hanessian, Beaconsfield (CA); Luciana Auzzas, Sassari (IT); Andreas Larsson, Umea (SE)

(73) Assignee: SIGMA-TAU INDUSTRIE FARMACEUTICHE RIUNITE S.P.A., ROME (IT)

(21) Appl. No.: 13/502,851

(22) PCT Filed: Sep. 21, 2010
(86) PCT No.: PCT/EP2010/063894

§ 371 Date: Jun. 21, 2012

(30) Foreign Application Priority Data
Oct. 21, 2009 (EP) 09173603.3

(51) Int. Cl. A61K 31/395; A61P 25/00; C07D 498/04; A61P 33/02; C07D 273/02; A61K 31/407; A61P 35/00; A61P 29/00

(52) U.S. Cl. 514/410; 540/455; 514/450

(57) ABSTRACT

The present invention relates to novel amide compounds of formula I, and their use as anti-tumoral and pro-apoptotic agents. The invention includes the use of such compounds in medicine, in relation to cancer disease as well as other diseases where an inhibition of HDAC6 is responsive, and the pharmaceutical composition containing such compounds.

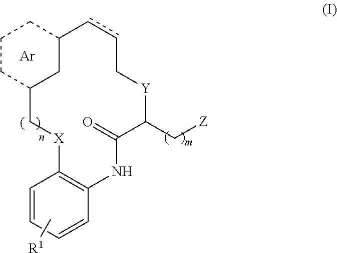

### FIELD OF THE INVENTION

The present invention relates to novel amide compounds and their use as anti-tumoral and pro-apoptotic agents. The invention includes the use of such compounds in medicine, in relation to cancer disease, inflammatory diseases, neuronal diseases, parasite infections (e.g., Plasmodium infection), as well as other diseases where an inhibition of HDAC6 is responsive, and the pharmaceutical composition containing such compounds.

### BACKGROUND OF THE INVENTION

Histone deacetylases (HDACs) are a family of enzymes found in numerous organisms among which bacteria, fungi, plants, and animals. Such enzymes catalyze the removal of acetyl groups from ε-N-acetylated lysine residues of various protein substrates including histones, transcription factors, α-tubulin, and nuclear importers.

Up to date eighteen HDAC isoforms have been characterized HDACs. They are classified in four different families with regard to their DNA sequence similarity and their biological role within the cells.

HDAC1, HDAC2, HDAC8 and HDAC3 are members of class-I. The first three isoforms are primarily found in the nucleus; meanwhile HDAC3 is also found in the cytoplasm or membrane-associated.

HDAC4, HDAC5, HDAC6, HDAC7, HDAC9 and HDAC10 form class-II. This class has been further divided in two sub-classes, class IIa (HDAC4, 5, 7 and 9) and class II-b (HDAC6 and 10). Class-II enzymes are expressed in a limited number of cell types and either shuttle between the nucleus and cytoplasm (class-IIa), or are mainly cytoplasmic (class-IIb) (Yang X. J., et al., Mol. Cell. Biol., 2005, 25, 2873).

Class-IV comprises only one member (HDAC11), meanwhile class-III, also called sirtuins, is composed of NAD⁺ dependent enzymes. The common feature of classes I, II and IV enzymes resides in their zinc dependent nature. HDAC inhibitors (HDACi) have been shown to be potent inducers of growth arrest, differentiation and apoptotic cell death of transformed cells in vitro and in vivo.

HDAC inhibition was also shown to lead to the reduction of inflammation in models of autoimmune and inflammatory diseases (Leoni F., et al., Proc. Natl. Acad. Sci., 2002, 99, 2995).

One of the first compounds to have been documented as HDACi was the well known anti-epileptic valproic acid, which inhibits all isoforms.

Once recognized the important role of this family of enzymes in the development of cancer, many efforts directed to find potent HDACi were undertaken by numerous academic groups as well as by pharmaceutical companies.

Vorinostat, originally known as SAHA (suberoylanilide hydroxamic acid), was the first-in-class small molecule hydroxamate derivative HDACi to have been approved by the FDA to treat a rare cancer, cutaneous T-cell lymphoma (Grant S., et al., Nature Rev. Drug Discov., 2007, 6, 21). SAHA is a potent non-selective HDACi inhibiting classes I and II as the vast majority of HDAC inhibitors currently in clinical trials (Paris M., et al., J. Med. Chem., 2008, 51, 1505)

Even though it has been demonstrated that the various HDAC isoforms have distinct biological functions and are recruited to specific regions of the genome, the exact physiological role of HDACs in cells is far from being completely elucidated. However, some progresses have been made lately in attributing important functions to some of them, such as their involvement in cardiac development (HDAC5 and 6), in neuronal cell death (HDAC4), mitosis (HDAC3), contractile capacity of smooth muscle cells (HDAC8), or cardiomyocyte differentiation (HDAC9).

Besides helping to unravel the complex physiology of the eleven zinc-dependent HDAC isoforms, the design of specific HDACi would contribute to the development of safer drugs. Actually, according to their structures, the various families of inhibitors can be grouped, according to their structures in four main groups (i.e., short chain fatty acids (e.g., butyrate, valproic acid), hydroxamates (e.g., SAHA, trichostatin, LAQ-824), cyclic derivatives (e.g., romidepsin), and benzamide (e.g., MS-275)).

Some clinical trials involving combination therapies have been conducted, to assess the efficacy of broad spectrum HDACi in combination with standard chemotherapeutic agents, (e.g., docetaxel and vorinostat), in patients with advanced and relapsed lung, bladder, or prostate cancer (clinical trial NCT00565227).

There is increasing evidence that HDAC6 plays a role in cancer cells and may be a target for drug development. HDAC6 presents the unique feature to possess two functional catalytic deacetylase domains and a carboxy terminal binding-of-ubiquitin zinc finger domain.

Tessier P. et al., nicely summarized the few selective HDAC6 inhibitors that have been recently reported in the literature (Tessier P. et al., Bioorg. Med. Chem. Lett., 2009, 19, 5684).

Targeted inhibition of HDAC6 provokes acetylation of HSP90 and disruption of its chaperone function with its client proteins Bcr-Abl (Bali P., et al., J. Biol. Chem., 2005, 280, 26729) leading to antimetastatic and antiangiogenic effects (Haggarty, S. J., et al., Proc. Natl. Acad. Sci., 2003, 100, 4389). Rodriguez-Gonzalez A., et al., further documented the potential involvement of HDAC6 in the development of metastasis originating from breast cancer (Rodriguez-Gonzalez A., et al., Cancer Res., 2008, 68, 2557).

HDAC has been hypothesized as a potential target for the treatment of parasite infections (e.g., Plasmodium infection) some thirteen years ago. However, the importance of the HDAC6 subtype has recently been further clarified (Chen Y., et al., J. Med. Chem., 2008, 51, 3437).

HDAC6 inhibition has been reported to be strongly involved in neuroprotection (Dompierre, J. P.; et al., J. Neurosci., 2007, 27, 3571). HDAC6 targeting blocks EGF induced nuclear translocation of β-catenin and c-myc expression in colon carcinoma cells.

It has been shown that HDAC6 was also able to deacetylate tubulin, therefore HDAC6 inhibitors leading to stable hyperacetylated tubulin may potentially be useful in the treatment of solid tumours and haematopoietic malignancies through potentiation of the activity of taxane agents such as docetaxel or paclitaxel (Yu Z., et al., EMBO J:, 2003, 22, 1168).

Very recently, HDAC6 was shown to be involved in epithelial-mesenchymal transition (EMT) leading to tumour progression and tissue fibrosis by influencing the TGF-β—SMAD3 cascade (Shan B., et al., J. Biol. Chem., 2008, 283, 21065). It is noteworthy that EMT and mesenchymal-to-epithelial transition (MET), have been reported to facilitate metastasis in a multitude of cancers among which kidney cancer, renal cancer, and bladder cancer (Chaffer C. L., et al., Cancer Res., 2006, 66, 11271).

A few selective HDAC6 inhibitors have been reported lately (Heltweg, B., et al., J. Med. Chem., 2004, 47, 5235; Yukihiro I., et al., Curr. Pharm. Des., 2008, 14, 529; Kozikowski A. P:, et al., J. Med. Chem., 2008, 51, 4370). Cyclic tetrapeptide derivatives wherein one α-amino acid was replaced by a β-amino acid have also been disclosed as potent HDAC6 inhibitors (Olsen C. A., et al., J. Med. Chem., 2009, 52, ASAP).

It has been reported lately that specific inhibition of HDAC6 leads to blockage of fibroblast invasion motility (Dong-Anh Tran A., et al., J. Cell Science, 2007, 120, 1469). This finding opens the way to new strategies to treat inflammation diseases such as arthritis and/or rheumatoid arthritis. This can be partially confirmed by the sponsorship Acetylon Pharmaceuticals just received to develop selective HDAC6 inhibitors to treat rheumatoid arthritis.

WO/2008/110583 filed in the name of the Applicant reported new macrocyclic derivatives presenting selective inhibitory activity against HDAC6.

However, in order to further increase the chance of finding an adequate treatment against cancer diseases, additional potent and selective inhibitors are still highly desired.

### DESCRIPTION OF THE INVENTION

It has now been found that new non-peptidic macrocycle derivatives are endowed with potent and selective inhibitory activity against the isoform HDAC6.

The invention provides compounds of formula (I) or a salt, hydrate or solvate thereof, in the preparation of a composition for inhibition of HDAC6 activity:

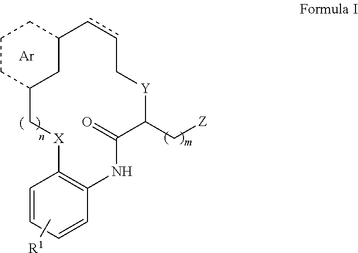

wherein,

### X is CONH or NHCO;

### Y is O, NH, NHCO or CONH;

### Z is CONHOH, SH, SAc, COCH₃ or CO₂H;

Ar is C₆-aryl or C₅-C₁₀-heteroaryl, wherein said aryl or heteroaryl can be optionally substituted with 1 to 4 groups chosen from the group consisting of C₁-C₃-alkyl, hydroxyl, alkoxy, amino or alkylamino;
R¹ is H, CONHR², NHR², amino-(C₁-C₂)-alkyl or (C₁-C₂)-alkyl-amino-(C₁-C₂)-alkyl;
R² is H or C₁-C₃-alkyl;
m is an integer comprised between 4 and 6;
n is an integer comprised between 0 and 1;
their tautomers, their geometrical isomers, their optically active forms such as enantiomers, diastereomers and their racemate forms, as well as their pharmaceutically acceptable salts thereof.

An embodiment of this invention is that of compounds of formula I, for use as medicaments.

In a further embodiment, said medicament is used for treating a subject affected by cancer diseases.

The invention furthermore provides a process for the preparation of compounds of formula I, which can be prepared by conventional synthetic methods and are described underneath.

Compounds of formula I, wherein X is NHCO, with the nitrogen atom linked to the phenyl moiety, R¹, Y, Z, Ar, m and n being as defined above, can be obtained by reacting compounds of formula II

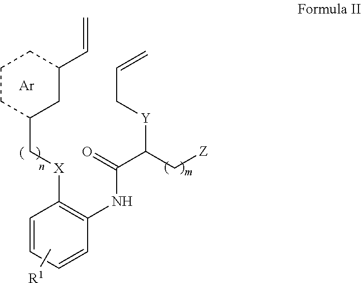

wherein R¹, Y, Z, Ar, m and n are as defined previously and X is NHCO, with the nitrogen atom linked to the phenyl moiety, with Grubbs' second generation or Hoveyda-Grubbs' second generation catalyst (Hong S. H:, et al., J. Am. Chem. Soc., 2006, 128, 3508) in an aprotic solvent such as toluene or dichloroethane at reflux temperature for up to 48 hours.

Compounds of formula II as above defined, can be obtained by reacting compounds of formula III,

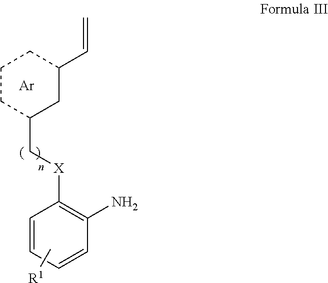

wherein R¹, Ar and n are as defined previously and X is NHCO, with the nitrogen atom linked to the phenyl moiety, with compounds of formula IV,

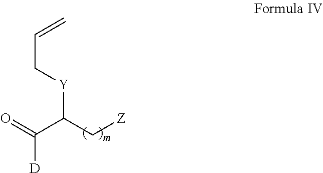

wherein Y, Z and m are as defined previously and D is OH in the presence of DIPEA and of a coupling agent such as HOBt, HOAt, EDC, or 3-(diethoxyphosphoryloxy)-1,2,3-benzotriazin-4(3H)-one (Li H., et al., Org. Lett., 1999, 1, 91) in an aprotic solvent such as THF or DCM.

Alternatively, compounds of formula II as above defined, can be obtained by reacting compounds of formula III as above defined, with compounds of formula IV, wherein Y, Z and m are as defined previously and D is Cl, in the presence of DIPEA in an aprotic solvent such as THF or DCM.

Compound of formula IV as above defined with D being OH, can be obtained following the protocol described in WO08110583.

In all said transformations, any interfering reactive group can be protected and then deprotected according to well-established procedures described in organic chemistry (see for example: Greene T. W. and P. G. M. Wuts “Protective Groups in Organic Synthesis”, J. Wiley & Sons, Inc., 3rd Ed., 1999) and well known to those skilled in the art.

All said transformations are only examples of well-established procedures described in organic chemistry (see for example: March J., “Advanced Organic Chemistry”, J. Wiley & Sons, Inc., 4th Ed., 1992) and well known to those skilled in the art.

The term “alkyl” refers to linear or branched alkyl groups having from 1 to 20 carbon atoms, or preferably, 1 to 12 carbon atoms or even more preferably 1 to about 6 carbon atoms.

The terms “C₁-Cₓ-alkyl” and “C₁-Cₓ-cycloalkyl”, wherein x is an integer comprised between 1 and 6, alone or encompassed in a more complex structure, refers to linear or branched alkyl or cycloalkyl groups having from 1 to 6 carbon atoms respectively.

The term “alkoxy” refers to the group —O—R where R includes “C₁-C₆ alkyl”, “C₃-C₁₀ cycloalkyl”.

The term “alkylamino” refers to amino groups which are substituted with alkyl groups.

The term “amino-(C₁-C₂)-alkyl” refers to alkyl groups containing one or two carbon atoms which are substituted with an amino residue.

The term “(C₁-C₂)-alkyl-amino-(C₁-C₂)-alkyl” refers to alkyl groups containing one or two carbon atoms which are substituted with an amino residue which is itself substituted with a C₁-C₂-alkyl moiety.

The term “C₅-C₁₀-heteroaryl” refers to a monocyclic heteroaromatic, or a bicyclic fused-ring heteroaromatic group. Particular examples of heteroaromatic groups include pyridyl, indolyl, pyrrolyl, furyl, thienyl, imidazolyl, oxazolyl, isoxazolyl, thiazolyl, isothiazolyl, pyrazolyl and benzofuryl.

“Pharmaceutically acceptable salts or complexes” refers to salts or complexes of the below identified compounds of formula (I), that retain the desired biological activity. Examples of such salts include, but are not restricted to acid addition salts formed with inorganic acids (e.g. hydrochloric acid, hydrobromic acid, sulfuric acid, phosphoric acid, nitric acid, and the like), and salts formed with organic acids such as acetic acid, oxalic acid, tartaric acid, succinic acid, malic acid, fumaric acid, maleic acid, ascorbic acid, benzoic acid, tannic acid, pamoic acid, alginic acid, polyglutamic acid, naphthalene sulfonic acid, toluene sulfonic acid, naphthalene disulfonic acid, methanesulfonic acid, and poly-galacturonic acid.

We have found that the derivatives (I) and their pharmaceutically acceptable salts, prepared according to the invention, are useful agents for the treatment of disease states, disorders and pathological conditions mediated by HDAC6; in particular for the treatment of cancer diseases and inflammatory diseases.

The pharmaceutical compositions will contain at least one compound of Formula (I) as an active ingredient, in an amount such as to produce a significant therapeutic effect. The compositions covered by the present invention are entirely conventional and are obtained with methods which are common practice in the pharmaceutical industry, such as, those illustrated in Remington's Pharmaceutical Science Handbook, Mack Pub. N.Y.—last edition. According to the administration route chosen, the compositions will be in solid or liquid form, suitable for oral, parenteral or topical administration. The compositions according to the present invention contain, along with the active ingredient, at least one pharmaceutically acceptable vehicle or excipient. These may be particularly useful formulation coadjuvants, e.g. solubilising agents, dispersing agents, suspension agents, and emulsifying agents.

Generally, the compounds of this invention are administered in a “therapeutically effective amount”. The amount of the compound actually administered will typically be determined by a physician, in the light of the relevant circumstances, including the condition to be treated, the chosen route of administration, the actual compound administered, drug combination, age, body weight, response of the individual patient, the severity of the patient's symptoms, and the like. For any compound, the therapeutically effective dose can be estimated initially either in cell culture assays or in animal models, usually mice, rats, guinea pigs, rabbits, dogs, or pigs. The animal model may also be used to determine the appropriate concentration range and route of administration. Such information can then be used to determine useful doses and routes for administration in humans. In calculating the Human Equivalent Dose (HED) it is recommended to use the conversion table provided in Guidance for Industry and Reviewers document (2002, U.S. Food and Drug Administration, Rockville, Md., USA).

Generally, an effective dose will be from 0.01 mg/kg to 100 mg/kg, preferably 0.05 mg/kg to 50 mg/kg. For any compound, the therapeutically effective dose can be estimated initially either in cell culture assays or in animal models, usually mice, rats, guinea pigs, rabbits, dogs, or pigs. The precise effective dose for a human subject will depend upon the severity of the disease state, general health of the subject, age, weight, and gender of the subject, diet, time and frequency of administration, drug combination(s), reaction sensitivities, and tolerance/response to therapy. This amount can be determined by routine experimentation and is within the judgement of the clinician.

Compositions may be administered individually to a patient or may be administered in combination with other agents, drugs or hormones.

The medicament may also contain a pharmaceutically acceptable carrier, for administration of a therapeutic agent. Such carriers include antibodies and other polypeptides, genes and other therapeutic agents such as liposomes, provided that the carrier does not induce the production of antibodies harmful to the individual receiving the composition, and which may be administered without undue toxicity.

Suitable carriers may be large, slowly metabolised macromolecules such as proteins, polysaccharides, polylactic acids, polyglycolic acids, polymeric amino acids, amino acid copolymers and inactive virus particles.

A thorough discussion of pharmaceutically acceptable carriers is available in Remington's Pharmaceutical Sciences (Mack Pub. Co., N.J. 1991).

Pharmaceutically acceptable carriers in therapeutic compositions may additionally contain liquids such as water, saline, glycerol and ethanol.

Additionally, auxiliary substances, such as wetting or emulsifying agents, pH buffering substances, and the like, may be present in such compositions. Such carriers enable the pharmaceutical compositions to be formulated as tablets, pills, dragees, capsules, liquids, gels, syrups, slurries, suspensions, and the like, for ingestion by the patient.

Once formulated, the compositions of the invention can be administered directly to the subject. The subjects to be treated can be animals; in particular, human subjects can be treated.

The medicament of this invention may be administered by any number of routes including, but not limited to, oral, intravenous, intramuscular, intra-arterial, intramedullary, intrathecal, intraventricular, transdermal or transcutaneous applications, subcutaneous, intraperitoneal, intranasal, enteral, topical, sublingual, intravaginal or rectal means.

The compositions for oral administration may take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include refilled, pre-measured ampoules or syringes of the liquid compositions or pills, tablets, capsules or the like in the case of solid compositions. In such compositions, the compound of the invention is usually a minor component (from about 0.1 to about 50% by weight or preferably from about 1 to about 40% by weight) with the remainder being various vehicles or carriers and processing aids helpful for forming the desired dosing form. Dosage treatment may be a single dose schedule or a multiple dose schedule. As above disclosed, the compounds of the present invention are useful as medicaments due to their HDAC6 inhibiting properties for the treatment of disorders where such inhibition result in improving the health of the patient. In particular, patients suffering from cancer and inflammatory diseases.

The compositions in question may, together with the compounds of formula (I), contain known active principles.

A further object of the invention is a process for the preparation of pharmaceutical compositions characterised by mixing one or more compounds of formula (I) with suitable excipients, stabilizers and/or pharmaceutically acceptable diluents.

An embodiment of this invention is that of compounds of formula (I) described earlier, wherein R¹ represents H.

A preferred embodiment of this invention is that of compounds of formula (I) described earlier, wherein Z represents CONHOH.

Still another embodiment of the present invention consists of the compounds selected from the group consisting of:
6-((Z)—(S)-19-methoxy-2,11-dioxo-13-oxa-3,10-diaza-tricyclo[15.3.1.0*4,9]henicosa-1(21),4,6,8,15,17,19-heptaen-12-yl)-hexanoic hydroxamic acid, 6-((Z)—(R)-19-methoxy-2,11-dioxo-13-oxa-3,10-diaza-tricyclo[15.3.1.0*4,9*]henicosa-1(21),4,6,8,15,17,19-heptaen-12-yl)-hexanoic hydroxamic acid, (S)-2-(6,18-dioxo-5,6,7,9,10,11,12,13,18,19-decahydrobenzo[5,6][1,4,7]-oxadiazacyclo-tetradecino[10,9-b]indol-7-yl)-N-hydroxyacetamide, 6-(S)-(2,11-dioxo-13-oxa-3,10-diaza-tricyclo[15.3.1.0*4,9*]henicosa-1(21),4(9),5,7,17,19-hexaen-12-yl)-hexanoic acid hydroxyamide, 6-(S)-(19-methoxy-2,11-dioxo-13-oxa-3,10-diaza-tricyclo[15.3.1.0*4,9*]henicosa-1(21),4(9),5,7,17,19-hexaen-12-yl)-hexanoic acid, 6-(S)-(19-hydroxy-2,11-dioxo-3,10,13-triaza-tricyclo[15.3.1.0*4,9*]henicosa-1(21),4(9),5,7,17,19-hexaen-12-yl)-hexanoic acid; 6-(S)-(19-methoxy-3,11-dioxo-13-oxa-2,10-diaza-tricyclo[15.3.1.0*4,9*]henicosa-1(21),4(9),5,7,17,19-hexaen-12-yl)-hexanoic acid hydroxyamide, 6-(S)-(20-methoxy-3,12-dioxo-14-oxa-4,11-diaza-tricyclo[16.3.1.0*5,10*]docosa-1(22),5(10),6,8,18,20-hexaen-13-yl)-hexanoic acid hydroxyamide, 6-(S)-17-(acetylamino-methyl)-19-methoxy-2,11-dioxo-13-oxa-3,10-diaza-tricyclo[15.3.1.0*4,9*]henicosa-[(21),4(9),5,7,17,19-hexaen-12-yl]-hexanoic acid hydroxyamide, N-hydroxy-6-(S)-(13-methoxy-6,18-dioxo-5,6,7,9,10,11,12,13,18,19-decahydroindolo[2,3-i][4,1,12]benzoxadiazacyclotetradecin-7-yl)hexanamide, N-hydroxy-6-(S)-(13-methoxy-6,18-dioxo-6,7,8,9,10,11,12,13,18,19-decahydro-5H-indolo[2,3-i][1,4,12]benzotriazacyclotetradecin-7-yl)hexanamide, 6-(S)-(13-methoxy-6,18-dioxo-5,6,7,9,10,11,12,13,18,19-decahydroindolo[2,3-i][4,1,12]benzoxadiazacyclotetradecin-7-yl)hexanoic acid, N-hydroxy-6-(S)-(16-methoxy-6,18-dioxo-5,6,7,9,10,11,12,13,18,19-decahydroindolo[2,3-i][4,1,12]benzoxadiazacyclotetradecin-7-yl)hexanamide, 6-(S)-(6,19-dioxo-5,6,7,9,10,11,12,17,18,19-decahydroindolo[2,3-h][4,1,11]benzoxadiazacyclotetradecin-7-yl)-N-hydroxyhexanamide, 6-(S)-(6,20-dioxo-6,7,9,10,11,12,13,18,19,20-decahydro-5H-indolo[2,3-i][4,1,12]benzoxadiazacyclopentadecin-7-yl)-N-hydroxyhexanamide, 6-(R)-(2,11-dioxo-13-oxa-3,10-diaza-tricyclo[15.3.1.0*4,9*]henicosa-1(21),4(9),5,7,17,19-hexaen-12-yl)-hexanoic acid hydroxyamide, 6-(R)(19-Methoxy-2,11-dioxo-13-oxa-3,10-diaza-tricyclo[15.3.1.0*4,9*]henicosa-1(21),4(9),5,7,17,19-hexaen-12-yl)-hexanoic acid, 6-(R)-(19-hydroxy-2,11-dioxo-3,10,13-triaza-tricyclo[15.3.1.0*4,9*]henicosa-1(21),4(9),5,7,17,19-hexaen-12-yl)-hexanoic acid; 6-(R)-(19-methoxy-3,11-dioxo-13-oxa-2,10-diaza-tricyclo[15.3.1.0*4,9*]henicosa-1(21),4(9),5,7,17,19-hexaen-12-yl)-hexanoic acid hydroxyamide, 6-(R)-(20-methoxy-3,12-dioxo-14-oxa-4,11-diaza-tricyclo[16.3.1.0*5,10*]docosa-1(22),5(10),6,8,18,20-hexaen-13-yl)-hexanoic acid hydroxyamide, 6-(R)-17-(acetylamino-methyl)-19-methoxy-2,11-dioxo-13-oxa-3,10-diaza-tricyclo[15.3.1.0*4,9*]henicosa-[(21),4(9),5,7,17,19-hexaen-12-yl]-hexanoic acid hydroxyamide, N-hydroxy-6-(R)-(13-methoxy-6,18-dioxo-5,6,7,9,10,11,12,13,18,19-decahydroindolo[2,3-i][4,1,12]benzoxadiazacyclotetradecin-7-yl)hexanamide, N-hydroxy-6-(R)-(13-methoxy-6,18-dioxo-6,7,8,9,10,11,12,13,18,19-decahydro-5H-indolo[2,3-i][1,4,12]benzotriazacyclotetradecin-7-yl)hexanamide, 6-(R)-(13-methoxy-6,18-dioxo-5,6,7,9,10,11,12,13,18,19-decahydroindolo[2,3-i][4,1,12]benzoxadiazacyclotetradecin-7-yl)hexanoic acid, N-hydroxy-6-(R)-(16-methoxy-6,18-dioxo-5,6,7,9,10,11,12,13,18,19-decahydroindolo[2,3-i][4,1,12]benzoxadiazacyclotetradecin-7-yl)hexanamide, 6-(R)-(6,19-dioxo-5,6,7,9,10,11,12,17,18,19-decahydroindolo[2,3-h][4,1,11]benzoxadiazacyclotetradecin-7-yl)-N-hydroxyhexanamide and 6-(R)-(6,20-dioxo-6,7,9,10,11,12,13,18,19,20-decahydro-5H-indolo[2,3-i][4,1,12]benzoxadiazacyclopentadecin-7-yl)-N-hydroxyhexanamide.

The following illustrated examples are by no means an exhaustive list of what the present invention intends to protect.

### EXAMPLES

### Abbreviations

DCM: dichloromethane
DEPBT: 3-(diethoxyphosphoryloxy)-1,2,3-benzotriazin-4(3H)-one
DIPEA: diisopropylethylamine
DMF: dimethylformamide
EtOAc: ethyl acetate
EtOH: ethanol
Et₂O: diethyl ether
KH₂PO₄: potassium dihydrogen phosphate
MeOH: methanol
MgSO₄: magnesium sulfate
NaHCO₃: sodium bicarbonate
NaH₂PO₄: sodium dihydrogen phosphate
NaHMDS: sodium hexamethyldisilazane
PDC: pyridinium dichromate
PyBrOP: bromo-tris-pyrrolidino phosphoniumhexafluorophosphate
RP-HPLC: reversed phase-high-performance liquid chromatography
SAc: thioacetyl

General Remarks All non-aqueous reactions were run in flame-dried glassware under a positive pressure of argon with exclusion of moisture from reagents and glassware using standard techniques for manipulating air-sensitive compounds. Anhydrous THF, toluene, Et₂O and DCM were obtained by filtration through drying columns (Solvent Delivery System); other solvents were distilled under positive pressure of dry argon before use and dried by standard methods. Commercial grade reagents were used without further purification. Flash chromatography was performed on 230-400 mesh silica gel with the indicated solvent systems. Thin layer chromatography was performed on pre-coated, glass-backed silica gel plates (Merck 60F₂₅₄). Visualization was performed under short-wavelength ultraviolet light and/or by dipping the plates in an aqueous H₂SO₄ solution of cerium sulfate/ammonium molybdate, potassium permanganate, or ethanolic solution of anisaldehyde, followed by charring with a heat gun. Routine nuclear magnetic resonance spectra were recorded on ARX-400, AV-400 spectrometers (Bruker) at 400, 100 and 75 MHz. Low- and high-resolution mass analyses were performed on AEI-MS 902 or MS-50 spectrometers using electrospray (ES) techniques. LCMS analyses were performed on a LC-Gilson apparatus (Autoinjector model 234, Pump 322), ThermoFinnigan LCQ Advantage MS and TSP UV6000 interface. Optical rotations were measured with a Perkin-Elmer 341 polarimeter at ambient temperature, using a 100 mm cell with a 1 ml capacity and are given in units of 10⁻¹ deg cm² g⁻¹.

Example 1 has been synthesized following the procedure as described in scheme 1.

### Example 1

### 6-((Z)—(S)-19-methoxy-2,11-dioxo-13-oxa-3,10-diaza-tricyclo[15.3.1.0*4,9*]henicosa-1(21),4,6,8,15,17,19-heptaen-12-yl)-hexanoic hydroxamic acid

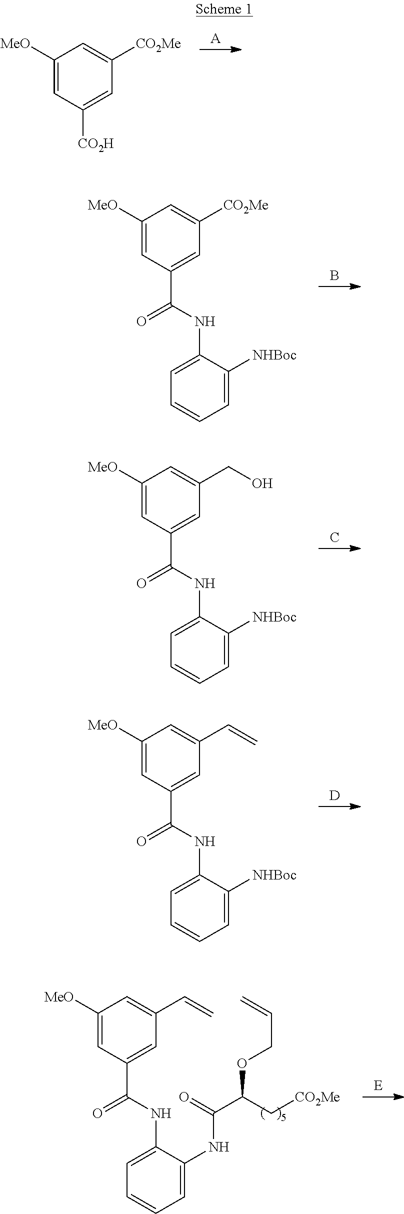

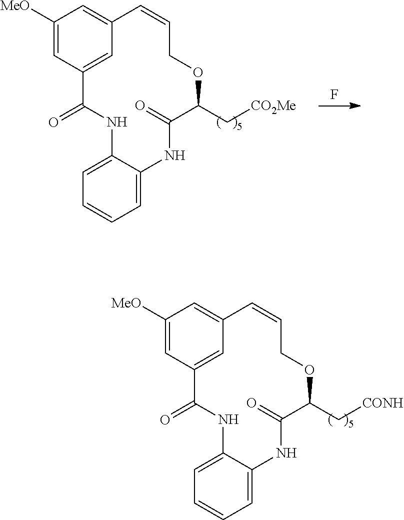

STEP A: N-(2-tert-butoxycarbonylamino-phenyl)-5-methoxy-isophthalamic acid methyl ester

DEPBT (996 mg, 3.33 mmol) and DIPEA (580 μl, 3.33 mmol) were added to a solution of 5-methoxy-isophthalic acid monomethyl ester (350 mg, 1.67 mmol) (Zhao H., et al., Synth. Comm., 2001, 31, 1921) in THF (8.5 ml). The resulting mixture was stirred for 20 min before adding a solution of tert-butyl (2-aminophenyl)carbamate (443 mg, 2.13 mmol) in THF (8.5 ml). The stirring was maintained for 18 hours, then quenched with sat. NH₄Cl (aq.), and extracted with EtOAc. The organic phase was washed with sat. NaHCO₃ (aq.) and brine, dried over MgSO₄, and concentrated under vacuum. Purification by flash chromatography (7:3 Hexanes/EtOAc) gave the desired N-(2-tert-butoxycarbonylamino-phenyl)-5-methoxy-isophthalamic acid methyl ester as a white solid.

Yield: 97%, 645 mg.

¹H-NMR (CDCl₃, 400 MHz) δ: 9.39 (bs, 1H), 8.18 (s, 1H), 7.77-7.72 (m, 2H), 7.71-7.67 (m, 1H), 7.31-7.27 (m, 1H), 7.20-7.11 (m, 2H), 7.03 (s 1H), 3.94 (s, 3H), 3.90 (s, 3H), 1.50 (s, 9H).

¹³C-NMR (CDCl₃, 100 MHz) δ: 166.2, 164.6, 159.9, 154.6, 135.9, 131.7, 130.2, 130.2, 126.1, 125.7, 125.6, 124.4, 120.1, 118.1, 118.0, 81.3, 55.7, 52.3, 28.2.

MS (ESI) m/z: 401.2 (M+H).

STEP B: [2-(3-hydroxymethyl-5-methoxy-benzoylamino)-phenyl]carbamic acid tert-butyl ester

DIBAL-H (1 M in DCM, 3.96 ml) was added dropwise to a solution of N-(2-tert-butoxycarbonylamino-phenyl)-5-methoxy-isophthalamic acid methyl ester in anhydrous THF (15 ml) at −78° C. The resulting solution was allowed to warm up to 0° C., and then quenched with sat. Rochelle salt (aq.). After stirring for one 1 hour, the mixture was extracted with Et₂O, dried over MgSO₄, and concentrated under vacuum. Purification by flash chromatography (1:1 Hexanes/EtOAc) gave [2-(3-hydroxymethyl-5-methoxy-benzoylamino)-phenyl]carbamic acid tert-butyl ester as a colourless oil.

Yield: 78%, 383 mg.

¹H-NMR (CDCl₃, 400 MHz) δ: 9.19 (bs, 1H), 7.68-7.62 (m, 1H), 7.39 (s, 2H), 7.34-7.29 (m, 1H), 7.20-7.13 (m, 2H), 7.07 (s, 2H), 4.63 (d, J=4.9 Hz, 2H), 3.83 (s, 3H), 2.80 (bs, 1H), 1.50 (s, 9H).

MS (ESI) m/z: 373.2 (M+H).

STEP C: [2-(3-methoxy-5-vinyl-benzoylamino)-phenyl]-carbamic acid tert-butyl ester

A solution of [2-(3-hydroxymethyl-5-methoxy-benzoylamino)-phenyl]-carbamic acid tert-butyl ester (383 mg, 1.03 mmol) and PDC (580 mg, 1.54 mmol) in DCM (20 ml) was stirred at RT under an argon atmosphere for 18 h. The reaction mixture was then filtered through a pad of silica gel/Celite®, and rinsed with EtOAc. The filtrate was concentrated under vacuum and purified by flash chromatography (hexanes/EtOAc 7:3), to afford the pure aldehyde intermediate as a white solid.

Yield: 92%, 349 mg.

¹H-NMR (CDCl₃, 400 MHz) δ: 10.00 (s, 1H), 9.60 (bs, 1H), 8.00 (s, 1H), 7.80 (s, 1H), 7.71-7.66 (m, 1H), 7.56-7.52 (m, 1H), 7.26-7.22 (m, 1H), 7.17-7.08 (m, 3H), 3.90 (s, 3H), 1.50 (s, 9H).

¹³C-NMR (CDCl₃, 100 MHz) δ: 191.2, 164.2, 160.4, 154.7, 137.8, 136.4, 130.1, 130.1, 126.1, 125.7, 125.6, 124.3, 121.7, 119.9, 115.5, 81.4, 55.8, 28.2.

MS (ESI) m/z: 371.2 (M+H).

NaHMDS (1 M in THF, 1.29 ml) was added dropwise at 0° C. to a solution of methyl-triphenylphosphonium bromide (493 mg, 1.38 mmol) in anhydrous THF (7 ml) under argon. The resulting solution was stirred for 15 min, and a solution of the above aldehyde intermediate (341 mg, 0.92 mmol) in anhydrous THF (5 ml) was slowly added to get a yellow mixture which was allowed to warm up to RT. After 1 hour, the reaction was quenched by addition of H₂O, and extracted with DCM. The organic phase was dried over MgSO₄, concentrated under vacuum and purified by flash chromatography (8:2 Hexanes/EtOAc) to afford the desired [2-(3-methoxy-5-vinyl-benzoylamino)-phenyl]-carbamic acid tert-butyl ester as a colourless oil.

Yield: 90%, 305 mg.

¹H-NMR (CDCl₃, 400 MHz) δ: 9.31 (bs, 1H), 7.68-7.63 (m, 1H), 7.58 (s, 1H), 7.45 (s, 1H), 7.30-7.25 (m, 1H), 7.17-7.07 (m, 4H), 6.73 (dd, J=17.6, 11.0 Hz, 1H), 5.86 (d, J=17.6 Hz, 1H), 5.35 (d, J=11.0 Hz, 1H), 3.88 (s, 3H), 1.51 (s, 9H).

¹³C-NMR (CDCl₃, 100 MHz) δ: 165.5, 160.0, 154.6, 139.3, 135.8, 135.7, 130.4, 130.2, 125.9, 125.6, 125.6, 124.5, 117.6, 115.4, 115.3, 112.1, 81.1, 55.5, 28.2.

MS (ESI) m/z: 369.2 (M+H).

STEP D: (S)-7-allyloxy-7-[2-(3-methoxy-5-vinyl-benzoylamino)-phenylcarbamoyl]-heptanoic acid methyl ester

To a solution of HCl (4N in dioxane, 2 ml) was added [2-(3-methoxy-5-vinyl-benzoylamino)-phenyl]-carbamic acid tert-butyl ester (115 mg, 0.31 mmol) at 0° C. The reaction mixture was stirred at 0° C. for 4 hours before being concentrated under vacuum. The residue was repeatedly suspended in cyclohexane and concentrated under vacuum to give the desired 2-(3-methoxy-5-vinyl-benzoylamino)-phenyl-ammonium chloride as a crude solid that was used in the next step without further purification.

DEPBT (196 mg, 0.66 mmol) and DIPEA (168 μl, 0.97 mmol) were added to a solution of (S)-2-allyloxy-octanedioic acid 8-methyl ester (obtained as described in WO08110583 starting from (S)-7-allyloxy-8-hydroxy-octanoic acid methyl ester) (80 mg, 0.33 mmol) in THF (1.5 ml). The resulting reaction mixture was stirred for 20 min and a 5 ml THF solution of 2-(3-methoxy-5-vinyl-benzoylamino)-phenyl-ammonium chloride was added. The reaction mixture was stirred for 18 h and was quenched by addition of a saturated aqueous solution of NH₄Cl, and then extracted with EtOAc. The organic phase was washed with sat. NaHCO₃ (aq.) and brine, dried over MgSO₄, and concentrated under vacuum. Purification by flash chromatography (6:4 Hexanes/EtOAc) gave (S)-7-allyloxy-7-[2-(3-methoxy-5-vinyl-benzoylamino)-phenylcarbamoyl]-heptanoic acid methyl ester as a white solid.

Yield: 77%, 118 mg.

¹H-NMR (CDCl₃, 400 MHz) δ: 9.35 (s, 1H), 8.72 (s, 1H), 7.87-7.82 (m, 1H), 7.61 (s, 1H), 7.47-7.43 (m, 1H), 7.36-7.20 (m, 3H), 7.14-7.10 (m, 1H), 6.75 (dd, J=17.7, 10.9 Hz, 1H), 5.97-5.86 (m, 1H), 5.88 (d, J=17.7 Hz, 1H), 5.36 (d, J=10.9 Hz, 1H), 5.30 (dd, J=17.7, 1.5 Hz, 1H), 5.22 (dd, J=10.3, 1.3 Hz, 1H), 4.10 (dd, J=5.8, 1.3 Hz, 2H), 4.00 (t, J=4.9 Hz, 1H), 3.90 (s, 3H), 3.65 (s, 3H), 2.20 (t, J=7.5 Hz, 2H), 1.89-1.74 (m, 2H), 1.54-1.09 (m, 6H).

¹³C-NMR (CDCl₃, 100 MHz) δ: 174.0, 172.5, 165.0, 160.1, 139.3, 136.0, 135.7, 133.4, 131.4, 129.1, 126.9, 126.2, 126.0, 124.5, 118.5, 117.4, 115.7, 115.4, 111.8, 79.5, 71.7, 55.5, 51.4, 33.8, 32.4, 28.8, 24.6, 24.2.

MS (ESI) m/z: 495.2 (M+H).

STEP E: 64(Z)—(S)-19-methoxy-2,11-dioxo-13-oxa-3,10-diaza-tricyclo[15.3.1.0*4,9*]henicosa-1(21),4,6,8,15,17,19-heptaen-12-yl)-hexanoic acid methyl ester

Hoveyda-Grubbs' catalyst (13 mg, 0.021 mmol) was added to a solution of (S)-7-allyloxy-7-[2-(3-methoxy-5-vinyl-benzoylamino)-phenylcarbamoyl]-heptanoic acid methyl ester (103 mg, 0.21 mmol) in dichloroethane (210 ml), and the resulting solution was refluxed for 48 hours before being concentrated under vacuum. Purification by flash chromatography (6:4 Hexanes/EtOAc) afforded 6-((Z)—(S)-19-methoxy-2,11-dioxo-13-oxa-3,10-diaza-tricyclo[15.3.1.0*4,9*]henicosa-1(21),4,6,8,15,17,19-heptaen-12-yl)-hexanoic acid methyl ester as a white solid.

Yield: 25%, 24 mg.

¹H-NMR (CDCl₃, 400 MHz) δ: 8.58 (s, 1H), 8.24 (s, 1H), 8.11-8.06 (m, 1H), 7.54 (s, 1H), 7.45-7.41 (m, 1H), 7.36-7.30 (m, 1H), 7.21-7.17 (m, 1H), 7.14-7.08 (m, 1H), 6.91 (s, 1H), 6.77 (d, J=11.5 Hz, 1H), 6.02 (ddd, J=16.6, 10.0, 7.2 Hz, 1H), 4.47 (t, J=9.9 Hz, 1H), 4.07-3.98 (m, 1H), 3.93-3.85 (m, 1H), 3.89 (s, 3H), 3.71 (s, 3H), 2.38 (t, J=7.4 Hz, 2H), 1.97-1.86 (m, 2H), 1.77-1.35 (m, 6H).

MS (ESI) m/z: 467.2 (M+H).

STEP F: 6-((Z)—(S)-19-methoxy-2,11-dioxo-13-oxa-3,10-diaza-tricyclo[15.3.1.0*4,9*]henicosa-1(21),4,6,8,15,17,19-heptaen-12-yl)-hexanoic hydroxamic acid

HONH₂ (50% aq. solution, 20 μl, 0.32 mmol) was added to a solution of 64(Z)—(S)-19-methoxy-2,11-dioxo-13-oxa-3,10-diaza-tricyclo[15.3.1.0*4,9*]henicosa-1(21),4,6,8,15,17,19-heptaen-12-yl)-hexanoic acid methyl ester (10 mg, 0.02 mmol) in MeOH-THF (1:1 v/v, 1 ml) at 0° C., followed by careful addition of 1 N NaOH (214 μl, 0.21 mmol). The mixture was allowed to reach RT within 4 hours and was stirred overnight. The reaction mixture was cooled to 0° C. before adding EtOAc and 1 N HCl (7-8 equiv). The resulting mixture was partitioned between EtOAc and H₂O. The organic phase was washed three times with H₂O, then brine, and dried over MgSO₄. The solvents were removed by co-evaporation with cyclohexane under vacuum, to afford the desired 6-((Z)—(S)-19-methoxy-2,11-dioxo-13-oxa-3,10-diaza-tricyclo[15.3.1.0*4,9*]henicosa-1(21),4,6,8,15,17,19-heptaen-12-yl)-hexanoic hydroxamic acid.

Yield: quantitative.

¹H-NMR (CDCl₃, 400 MHz) δ: 10.33 (bs, 1H), 10.14 (bs, 1H), 9.33 (bs, 1H), 8.67 (bs, 1H), 7.98-7.95 (m, 1H), 7.73 (s, 1H), 7.37-7.31 (m, 2H), 7.29-7.27 (m, 1H), 7.26-7.20 (m, 1H), 7.11-7.07 (m, 1H), 6.79 (d, J=11.6 Hz, 1H), 6.05 (ddd, J=17.3, 9.9, 7.3 Hz, 1H), 4.49 (t, J=9.8 Hz, 1H), 4.10 (dd, J=9.4, 7.7 Hz, 1H), 3.98 (t, J=6.7 Hz, 1H), 3.84 (s, 3H), 1.99 (t J=7.2 Hz, 2H), 1.82-1.74 (m, 2H), 1.60-1.28 (m, 6H).

MS (ESI) m/z: 468.2 (M+H).

### Example 2

### 6-((Z)—(R)-19-methoxy-2,11-dioxo-13-oxa-3,10-diaza-tricyclo[15.3.1.0*4,9*]henicosa-1(21),4,6,8,15,17,19-heptaen-12-yl)-hexanoic hydroxamic acid

Example 2 was prepared according to the procedure described in example 1 using (R)-2-allyloxy-octanedioic acid 8-methyl ester in Step D instead of (S)-2-allyloxy-octanedioic acid 8-methyl ester.

Analytical data were in agreement to those of its corresponding enantiomer described in example 1.

Example 3 has been synthesized following the procedure as described in scheme 2.

### Example 3

### (S)-2-(6,18-dioxo-5,6,7,9,10,11,12,13,18,19-decahydrobenzo[5,6][1,4,7]-oxadiazacyclo-tetradecino[10,9-b]indol-7-yl)-N-hydroxyacetamide

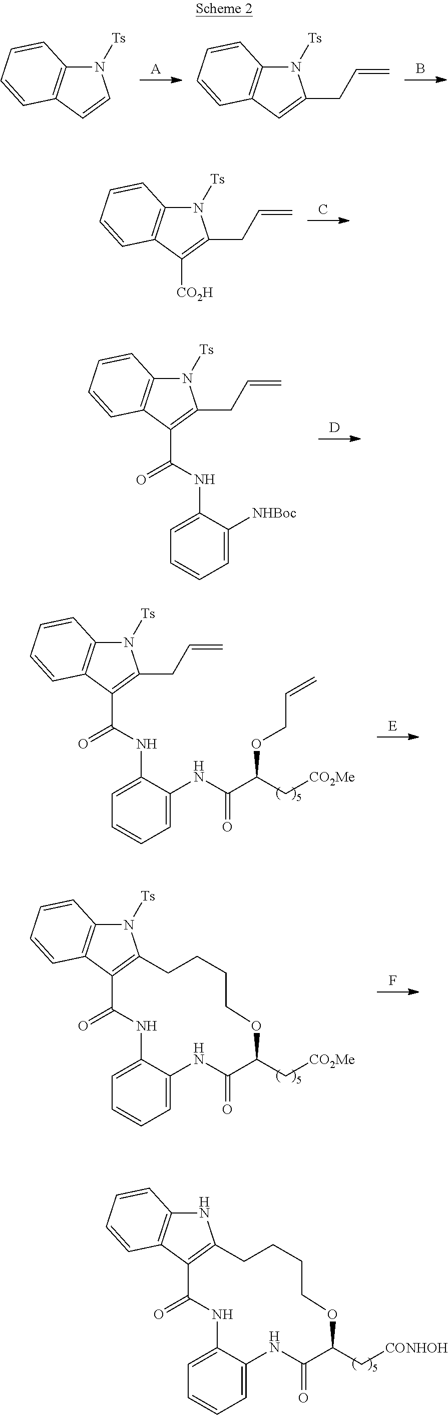

STEP A: 2-allyl-1-(toluene-4-sulfonyl)-1H-indole

t-BuLi (1.7 M in pentane, 22.8 ml, 38.70 mmol) was added dropwise to a solution of 1-tosyl-1H-indole (10.0 g, 36.86 mmol) in 210 ml Et₂O at −78° C. The resulting suspension was allowed to warm up to −40° C. within 1 hour before being cooled down to −78° C. I₂ (28.07 g, 110.58 mmol) was added in portions, and the mixture was allowed to reach RT overnight. The reaction was quenched by addition of NH₄Cl (aq. sat.) and the organic phase was washed repeatedly with Na₂S₂O₃ (aq. sat.), dried over MgSO₄, and concentrated under vacuum. Purification by flash chromatography (9:1 Hexanes/EtOAc) gave the iodinated product as a pale yellow solid.

Yield 81%, 11.86 g.

¹H-NMR (CDCl₃, 400 MHz) δ: 8.32-8.28 (m, 1H), 7.82-7.78 (m, 2H), 7.44-7.40 (m, 1H), 7.32-7.20 (m, 4H), 7.00 (s, 1H), 2.36 (s, 3H).

¹³C-NMR (CDCl₃, 100 MHz) δ: 145.2, 138.4, 135.2, 131.7, 129.7, 127.1, 124.8, 124.1, 123.7, 119.6, 115.4, 75.3, 21.6.

MS (ESI) m/z: 398.0 (M+H).

i-PrMgCl (2.0 M in THF, 20 ml, 40 mmol) was added dropwise at −20° C. to a solution of the above mentioned iodinated indole (7.95 g, 20 mmol) in 200 ml THF. The re-suiting solution was stirred for 2 hours. CuCl₂.2 LiCl (0.1 M in THF, 20 ml, 2.0 mmol) and allyl bromide (5.1 ml, 60 mmol) were sequentially added, and the resulting reaction mixture was allowed to reach RT. The reaction was quenched with brine and extracted EtOAC. The organic phase was dried over MgSO₄ and concentrated under vacuum. Purification by flash chromatography (95:5 Hexanes/EtOAc) gave 2-allyl-1-(toluene-4-sulfonyl)-1H-indole as a colourless oil.

Yield: 90%, 5.61 g.

¹H-NMR (CDCl₃, 400 MHz) δ: 8.22-8.16 (m, 1H), 7.71-7.64 (m, 2H), 7.46-7.41 (m, 1H), 7.32-7.18 (m, 4H), 6.43-6.40 (m, 1H), 6.07 (ddt, J=16.8, 10.1, 6.7 Hz, 1H), 5.24 (ddd, J=8.3, 3.1, 1.6 Hz, 1H), 5.22-5.20 (m, 1H) 3.80 (dd, J=6.7, 1.2 Hz, 2H), 2.36 (s, 3H).

¹³C-NMR (CDCl₃, 100 MHz) δ: 144.7, 140.1, 137.1, 136.1, 134.1, 129.8, 129.6, 126.3, 123.9, 123.4, 120.2, 117.8, 114.7, 109.3, 33.4, 21.5.

MS (ESI) m/z: 312.1 (M+H).

STEP B: 2-allyl-1-(toluene-4-sulfonyl)-1H-indole-3-carboxylic acid

TiCl₄ (1 M in DCM, 20.7 ml, 20.7 mmol) was added dropwise to a solution of 2-allyl-1-(toluene-4-sulfonyl)-1H-indole (2.15 g, 6.9 mmol) and dichloromethyl methyl ether (0.94 ml, 10.4 mmol) in DCM (70 ml) at −78° C. The resulting solution was stirred for 5 hours, quenched by addition of water, and extracted with DCM, and the organic phase was dried over MgSO₄ and concentrated under vacuum. Purification by flash chromatography (8:2 Hexanes/EtOAc) gave the intermediate aldehyde as a white solid.

Yield: 91%, 2.13 g.

¹H-NMR (CDCl₃, 400 MHz) δ: 10.30 (s, 1H), 8.33-8.27 (m, 1H), 8.22-8.16 (m, 1H), 7.77 (d, J=8.0 Hz, 2H), 7.42-7.34 (m, 2H), 7.26 (d, J=8.0 Hz, 2H), 6.11 (ddt, J=17.1, 10.1, 5.9 Hz, 1H), 5.16 (dd, J=10.1, 1.0, 1H), 5.10 (dd, J=17.1, 1.0 Hz, 1H), 4.26 (dt, J=5.9, 1.5 Hz, 2H), 2.37 (s, 3H).

¹³C-NMR (CDCl₃, 100 MHz) δ: 185.7, 148.9, 145.8, 135.8, 135.4, 134.3, 130.0, 126.7, 126.0, 125.6, 125.0, 121.4, 119.3, 117.6, 114.3, 29.4, 21.5.

MS (ESI) m/z: 340.1 (M+H).

NaClO2 (4.67 g, 41.2 mmol, 80%) was added to a solution of the above aldehyde (700 mg, 2.06 mmol) and NaH₂PO₄ (5.00 g, 41.66 mmol) in a mixture of t-BuOH:2-methyl-2-butene:H₂O (2:2:1, v/v, 100 ml). The resulting reaction mixture was stirred at RT for 24 h. After dilution with a 95/5 mixture of DCM:MeOH (200 ml), the solution was washed with brine, dried over MgSO₄, and concentrated under vacuum. The residue was suspended in a 0.5 M K₂CO₃ aqueous solution, and extracted with DCM. The aqueous phase was acidified with conc. HCl before being extracted with DCM, dried over MgSO₄, and concentrated under vacuum. The resulting oily residue was allowed to crystallize, and carefully rinsed with hexane to give 2-allyl-1-(toluene-4-sulfonyl)-1H-indole-3-carboxylic acid as colourless needles.

Yield: 94%, 689 mg.

¹H-NMR (CDCl₃, 400 MHz) δ: 8.26-8.20 (m, 2H), 7.75 (d, J=8.1 Hz, 2H), 7.41-7.34 (m, 2H), 7.26 (d, J=8.1 Hz, 2H), 6.07 (ddt, J=17.1, 10.2, 6.0 Hz, 1H), 5.18 (dd, J=17.1, 1.5 Hz, 1H), 5.11 (dd, J=10.2, 1.5 Hz, 1H), 4.40 (d, J=6.0 Hz), 2.39 (s, 3H).

¹³C-NMR (CDCl₃, 100 MHz) δ: 169.5, 148.2, 145.6, 135.9, 135.7, 134.4, 130.0, 127.3, 126.7, 125.1, 124.6, 122.0, 117.0, 114.6, 110.9, 30.5, 21.6.

MS (ESI) m/z: 356.1 (M+H).

STEP C: (2-{[2-allyl-1-(toluene-4-sulfonyl)-1H-indole-3-carbonyl]-amino}-phenyl)-carbamic acid tert-butyl ester

PyBrOP (555 mg, 1.20 mmol) and DIPEA (553 μl, 3.20 mmol) were sequentially added to a solution of 2-allyl-1-(toluene-4-sulfonyl)-1H-indole-3-carboxylic acid (282 mg, 0.80 mmol) and tert-butyl (2-aminophenyl)carbamate (198 mg, 0.90 mmol) in CHCl₃ (1.2 ml) at RT. The resulting reaction mixture was stirred for 48 h before being diluted with CHCl₃ and washed with 5% NaHCO₃ (aq.), water, and brine. After drying over Na₂SO₄, the solution was concentrated under vacuum. Purification by flash chromatography (8:2 Hexanes/EtOAc) gave the desired (2-{[2-allyl-1-(toluene-4-sulfonyl)-1H-indole-3-carbonyl]-amino}-phenyl)-carbamic acid tert-butyl ester as a white solid.

Yield: 78%, 320 mg.

¹H-NMR (CDCl₃, 400 MHz) δ: 8.28-8.24 (m, 1H), 8.21 (s, 1H), 7.90-7.85 (m, 1H), 7.74 (d, J=8.4 Hz, 2H), 7.60-7.52 (m, 2H), 7.42-7.31 (m, 2H), 7.28-7.14 (m, 4H), 6.97 (bs, 1H), 6.19 (ddt, J=17.1, 10.2, 5.8 Hz, 1H), 5.19 (dd, J=10.2, 1.4 Hz, 1H), 5.15 (dd, J=17.1, 1.4 Hz, 1H), 4.19 (d, J=5.8 Hz, 2H), 2.38 (s, 3H), 1.42 (s, 9H).

¹³C-NMR (CDCl₃, 100 MHz) δ: 163.2, 153.9, 145.4, 140.4, 136.0, 135.8, 135.3, 131.0, 130.0, 129.8, 129.7, 127.0, 126.6, 125.5, 125.1, 125.0, 124.8, 124.4, 119.8, 117.2, 117.2, 114.9, 80.8, 30.7, 28.2, 21.5.

MS (ESI) m/z: 546.2 (M+H).

STEP D: (S)-7-allyloxy-7-(2-{[2-allyl-1-(toluene-4-sulfonyl)-1H-indole-3-carbonyl]-amino}-phenylcarbamoyl)-heptanoic acid methyl ester

HCl (4N in dioxane, 1.5 ml) was added to (2-{[2-allyl-1-(toluene-4-sulfonyl)-1H-indole-3-carbonyl]-amino}-phenyl)-carbamic acid tert-butyl ester (120 mg, 0.22 mmol) at 0° C., and the resulting reaction mixture was stirred at 0° C. for 4 hours before being concentrated under vacuum. The obtained residue was repeatedly suspended in cyclohexane and concentrated under vacuum, to afford the desired 2-{[2-allyl-1-(toluene-4-sulfonyl)-1H-indole-3-carbonyl]-amino}-phenyl-ammonium chloride as a crude solid that was used in the next step without further purification. DEPBT (145 mg, 0.48 mmol) and DIPEA (123 μl, 0.70 mmol) were added to a solution of (S)-2-allyloxy-octanedioic acid 8-methyl ester ([α]²⁰_{D}=−13.3 (c 0.9, CHCl₃); 60 mg, 0.242 mmol) in THF (1 ml). The resulting solution was stirred for 20 min before the addition of a solution of 2-{[2-allyl-1-(toluene-4-sulfonyl)-1H-indole-3-carbonyl]-amino}-phenyl-ammonium chloride in THF (1 ml). The reaction mixture was stirred for 18 h, and was quenched by addition of sat. NH₄Cl (aq.), and extracted with EtOAc. The organic phase was washed with sat. NaHCO₃ (aq.) and brine, dried over MgSO₄ and concentrated under vacuum. Purification by flash chromatography (6:4 Hexanes/EtOAc) afforded the desired (5)-7-allyloxy-7-(2-{[2-allyl-1-(toluene-4-sulfonyl)-1H-indole-3-carbonyl]-amino}-phenylcarbamoyl)-heptanoic acid methyl ester as a white solid.

Yield: 78%, 115 mg.

¹H-NMR (CDCl₃, 400 MHz) δ: 8.90 (s, 1H), 8.34 (s, 1H), 8.24 (d, J=8.1 Hz, 1H), 7.87 (d, J=7.8 Hz, 1H), 7.74 (d, J=8.4 Hz, 2H), 7.63-7.59 (m, 1H), 7.55-7.49 (m, 1H), 7.41-7.23 (m, 6H), 6.16 (ddt, J=17.1, 10.2, 5.8 Hz, 1H), 5.77 (ddt, J=17.1, 10.6, 5.6 Hz, 1H), 5.19-5.10 (m, 3H), 4.99 (dd, J=10.4, 0.9 Hz, 1H), 4.28-4.14 (m, 2H), 4.08-3.92 (m, 2H), 3.87 (dd, J=6.6, 4.7 Hz, 1H), 3.66 (s, 3H), 2.38 (s, 3H), 2.23 (t, J=7.5 Hz, 2H), 1.76-1.66 (m, 2H), 1.54-1.44 (m, 2H), 1.39-1.30 (m, 2H), 1.24-1.13 (m, 2H).

¹³C-NMR (CDCl₃, 100 MHz) δ: 173.7, 171.7, 162.8, 145.1, 140.6, 135.6, 135.4, 135.0, 133.0, 130.0, 129.7, 129.6, 126.5, 126.4, 126.3, 126.1, 125.2, 124.8, 124.7, 124.0, 119.6, 117.7, 116.8, 116.6, 114.5, 79.4, 71.3, 51.1, 33.5, 32.2, 30.4, 28.4, 24.2, 24.1, 21.2.

MS (ESI) m/z: 672.3 (M+H).

STEP E: (S)-methyl-6(6,18-dioxo-13-tosyl-5,6,7,9, 10, 11,12,13,18,19-decahydro-benzo[5,6][1,4,7]oxadiazacyclotetradecino[10,9-b]indol-7-yl)hexanoate

A solution of Grubbs' second generation catalyst (13 mg, 0.023 mmol) and (S)-7-allyloxy-7-(2-{[2-allyl-1-(toluene-4-sulfonyl)-1H-indole-3-carbonyl]-amino}-phenylcarbamoyl)-heptanoic acid methyl ester (103 mg, 0.15 mmol) in dichloroethane (150 ml) was refluxed for 18 h before concentrated in vacuum. Purification by flash chromatography (6:4 Hexanes/EtOAc) gave 71 mg of the desired alkene as a mixture of E and Z isomers. The mixture was stirred in MeOH containing 3% Pd on carbon (4.7 mg) under H₂ atmosphere (1 atm) for 5 hours at RT. After filtration through a pad of Celite® the desired (S)-methyl-6-(6,18-dioxo-13-tosyl-5,6,7,9,10,11,12,13,18,19-decahydro-benzo[5,6][1,4,7]oxadiazacyclotetradecino[10,9-b]indol-7-yl)hexanoate was obtained as a white solid.

Yield: 72%, 71 mg.

¹H-NMR (CDCl₃, 400 MHz) δ: 9.00 (s, 1H), 8.21 (d, J=7.5 Hz, 1H), 8.11-8.03 (m, 2H), 7.76-7.64 (m, 3H), 7.40-7.12 (m, 7H), 3.72 (t, J=4.6 Hz, 1H), 3.63 (s, 3H), 3.51-3.30 (m, 2H), 2.37 (s, 3H), 2.30 (t, J=7.3 Hz, 2H), 2.00-1.88 (m, 1H), 1.84-1.56 (m, 7H), 1.49-1.24 (m, 6H).

MS (ESI) m/z: 646.3 (M+H).

STEP F: (S)-2-(6,18-dioxo-5,6,7,9,10,11,12,13,18,19-decahydrobenzo[5,6][1,4,7]-oxadiazacyclotetradecino[10,9-b]indol-7-yl)-N-hydroxyacetamide

KH₂PO₄ (8 mg, 0.06 mmol) under an Ar atmosphere was added to a solution of (S)-methyl-6-(6,18-dioxo-13-tosyl-5,6,7,9,10,11,12,13,18,19-decahydro-benzo[5,6][1,4,7]oxadiazacyclotetradecino[10,9-b]indol-7-yl)hexanoate (13 mg, 0.02 mmol) in THF-MeOH (2:1 v/v, 360 μl). Sodium amalgam (68 mg, 0.30 mmol, 10% Na), was then added, and the resulting reaction mixture was stirred for 5 h at RT. Mercury was left decanted from the solution before pouring the latter into sat. NaHCO₃ (aq.). The resulting mixture was extracted with EtOAc. The organic phase was dried over MgSO₄ and concentrated under vacuum. The deprotected macrocyclic ester was dissolved in MeOH-THF (1:1 v/v, 1 ml) at 0° C., and HONH₂ (50% aq. solution, 20 μl, 0.32 mmol) was added, followed by careful addition of 1 N NaOH (214 μl, 0.21 mmol). The mixture was allowed to reach RT within 3-4 h, and was stirred overnight. The reaction mixture was cooled to 0° C. and EtOAc was added, followed by 1 N HCl (7-8 equiv). The resulting mixture was partitioned between EtOAc and H₂O. The organic phase was washed three times with H₂O, then with brine, and finally dried over MgSO₄. The solvents were removed by co-evaporation with cyclo-hexane under vacuum, to furnish the desired (S)-2-(6,18-dioxo-5,6,7,9,10,11,12,13,18,19-decahydrobenzo[5,6][1,4,7]-oxadiazacyclotetradecino[10,9-b]indol-7-yl)-N-hydroxyacetamide.

Yield: quantitative.

¹H-NMR (CDCl₃, 400 MHz) δ: 11.54 (s, 1H), 10.31 (bs, 1H), 9.55 (bs, 1H), 9.12 (bs, 1H), 8.65 (bs, 1H), 8.06-8.01 (m, 1H), 7.85-7.81 (m, 1H), 7.52-7.48 (m, 1H), 7.41-7.37 (m, 1H), 7.33-7.27 (m, 1H), 7.23-7.18 (m, 1H), 7.17-7.10 (m, 2H), 3.81-3.76 (m, 1H), 3.65-3.58 (m, 1H), 3.53-3.45 (m, 1H), 2.04-1.96 (m, 2H), 1.93 (t, J=7.3 Hz, 2H), 1.80-1.57 (m, 5H), 1.53-1.24 (m, 7H).

MS (ESI) m/z: 647.3 (M+H).

### BIOLOGICAL RESULTS

### Materials and Methods

### Cytotoxicity Assay:

Table 1 reports IC₅₀ data were obtained using human HDAC enzymes and a fluorogenic peptide as the substrate (10 μM), which was bound to a specific p53 fragment—residues 379-392: Arg-His-Lys-Lys(Ac) comprising an E-acetylated lysine side chain. The substrate was incubated with the eleven single HDAC purified enzymes. Upon its deacetylation, the fluorophore was released given rise to fluorescence emission. The latter was detected by a fluorimeter, and the IC₅₀ values of the compounds were determined by analyzing dose-response inhibition curves. TSA and SAHA were used as reference compounds.

TABLE 1

| | | | Example 1 | Example 2 | Example 3 |
|---|---|---|---|---|---|
| | SAHA | TSA | (S)-isomer | (R)-isomer | (R)-isomer |
| | (IC₅₀ nm) | (IC₅₀ nm) | (IC₅₀ nm) | (IC₅₀ nm) | (IC₅₀ nm) |
| HDAC1 | 258 | 7.1 | 864 | 1340 | 245 |
| HDAC2 | 921 | 22.9 | 3280 | 2530 | 584 |
| HDAC3 | 350 | 10.3 | 1114 | 885 | 331 |
| HDAC4 | 493 | 12.1 | 5333 | 3480 | 1690 |
| HDAC5 | 378 | 16.5 | 1660 | 1340 | 496 |
| HDAC6 | 28.6 | 0.4 | 59 | 75 | 4.4 |
| HDAC7 | 344 | 22.5 | 4300 | 2070 | 2230 |
| HDAC8 | 243 | 89.5 | 534 | 682 | 297 |
| HDAC9 | 316 | 38.1 | 2020 | 1140 | 602 |
| HDAC10 | 456 | 20.1 | 2290 | 1880 | 627 |
| HDAC11 | 362 | 15.2 | 948 | 1110 | 379 |

Compounds of examples 1 and 3 demonstrated a very high selectivity toward the other HDAC isoforms ranging from 9 to 90 and from 60 to 550 respectively. It was also surprising to note that the stereochemistry on the carbon atom in position 6 of the hexanoic chain had only little effect on the potency of the compound. This result was most unexpected, since among analogues bearing an ether bridge instead of the second amide moiety, the stereochemistry of the above mentioned centre was much more influent on the affinity of the molecule toward the various HDAC isoforms.

1. A compound having the general formula I 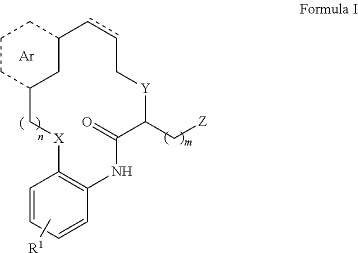
wherein,
X is CONH or NHCO;
Y is O, NH, NHCO or CONH;
Z is CONHOH, SH, SAc, COCH₃ or CO₂H;
Ar is C₆-aryl or C₅-C₁₀-heteroaryl, wherein said aryl or heteroaryl can be optionally substituted with 1 to 4 groups chosen from the group consisting of C₁-C₃-alkyl, hydroxyl, alkoxy, amino or alkylamino;
R¹ is H, CONHR², NHR², amino-(C₁-C₂)-alkyl or (C₁-C₂)-alkyl-amino-(C₁-C₂)-alkyl;
R² is H or C₁-C₃-alkyl;
m is an integer comprised between 4 and 6;
n is an integer comprised between 0 and 1;
their tautomers, their geometrical isomers, their optically active forms such as enantiomers, diastereomers and their racemate forms, as well as their pharmaceutically acceptable salts thereof.

2. A compound according to claim 1, wherein Z is CONHOH.

3. A compound according to claim 1 selected from the group consisting of:
6-((Z)—(S)-19-methoxy-2,11-dioxo-13-oxa-3,10-diaza-tricyclo[15.3.1.0*4,9*]henicosa-1(21),4,6,8,15,17,19-heptaen-12-yl)-hexanoic hydroxamic acid, 6-((Z)—(R)-19-methoxy-2,11-dioxo-13-oxa-3,10-diaza-tricyclo[15.3.1.0*4,9*]henicosa-1(21),4,6,8,15,17,19-heptaen-12-yl)-hexanoic hydroxamic acid, (S)-2-(6,18-dioxo-5,6,7,9,10,11,12,13,18,19-decahydrobenzo[5,6][1,4,7]-oxadiazacyclo-tetradecino[10,9-b]indol-7-yl)-N-hydroxyacetamide, 6-(S)-(2,11-dioxo-13-oxa-3,10-diaza-tricyclo[15.3.1.0*4,9*]henicosa-1(21),4(9),5,7,17,19-hexaen-12-yl)-hexanoic acid hydroxyamide, 6-(S)-(19-methoxy-2,11-dioxo-13-oxa-3,10-diaza-tricyclo[15.3.1.0*4,9*]henicosa-1(21),4(9),5,7,17,19-hexaen-12-yl)-hexanoic acid, 6-(S)-(19-hydroxy-2,11-dioxo-3,10,13-triaza-tricyclo[15.3.1.0*4,9*]henicosa-1(21),4(9),5,7,17,19-hexaen-12-yl)-hexanoic acid; 6-(S)-(19-methoxy-3,11-dioxo-13-oxa-2,10-diaza-tricyclo[15.3.1.0*4,9*]henicosa-1(21),4(9),5,7,17,19-hexaen-12-yl)-hexanoic acid hydroxyamide, 6-(S)-(20-methoxy-3,12-dioxo-14-oxa-4,11-diaza-tricyclo[16.3.1.0*5,10*]docosa-1(22),5(10),6,8,18,20-hexaen-13-yl)-hexanoic acid hydroxyamide, 6-(S)-17-(acetylamino-methyl)-19-methoxy-2,11-dioxo-13-oxa-3,10-diaza-tricyclo[15.3.1.0*4,9*]henicosa-[(21),4(9),5,7,17,19-hexaen-12-yl]-hexanoic acid hydroxyamide, N-hydroxy-6-(S)-(13-methoxy-6,18-dioxo-5,6,7,9,10,11,12,13,18,19-decahydroindolo[2,3-i][4,1,12]benzoxadiazacyclotetradecin-7-yl)hexanamide, N-hydroxy-6-(S)-(13-methoxy-6,18-dioxo-6,7,8,9,10,11,12,13,18,19-decahydro-5H-indolo[2,3-i][1,4,12]benzotriazacyclotetradecin-7-yl)hexanamide, 6-(S)-(13-methoxy-6,18-dioxo-5,6,7,9,10,11,12,13,18,19-decahydroindolo[2,3-i][4,1,12]benzoxadiazacyclotetradecin-7-yl)hexanoic acid, N-hydroxy-6-(S)-(16-methoxy-6,18-dioxo-5,6,7,9,10,11,12,13,18,19-decahydroindolo[2,3-i][4,1,12]benzoxadiazacyclotetradecin-7-yl)hexanamide, 6-(S)-(6,19-dioxo-5,6,7,9,10,11,12,17,18,19-decahydroindolo[2,3-h][4,1,11]benzoxadiazacyclotetradecin-7-yl)-N-hydroxyhexanamide, 6-(S)-(6,20-dioxo-6,7,9,10,11,12,13,18,19,20-decahydro-5H-indolo[2,3-i][4,1,12]benzoxadiazacyclopentadecin-7-yl)-N-hydroxyhexanamide, 6-(R)-(2,11-dioxo-13-oxa-3,10-diaza-tricyclo[15.3.1.0*4,9*]henicosa-1(21),4(9),5,7,17,19-hexaen-12-yl)-hexanoic acid hydroxyamide, 6-(R)(19-Methoxy-2,11-dioxo-13-oxa-3,10-diaza-tricyclo[15.3.1.0*4,9*]henicosa-1(21),4(9),5,7,17,19-hexaen-12-yl)-hexanoic acid, 6-(R)-(19-hydroxy-2,11-dioxo-3,10,13-triaza-tricyclo[15.3.1.0*4,9*]henicosa-1(21),4(9),5,7,17,19-hexaen-12-yl)-hexanoic acid; 6-(R)-(19-methoxy-3,11-dioxo-13-oxa-2,10-diaza-tricyclo[15.3.1.0*4,9*]henicosa-1(21),4(9),5,7,17,19-hexaen-12-yl)-hexanoic acid hydroxyamide, 6-(R)-(20-methoxy-3,12-dioxo-14-oxa-4,11-diaza-tricyclo[16.3.1.0*5,10*]docosa-1(22),5 (10),6,8,18,20-hexaen-13-yl)-hexanoic acid hydroxyamide, 6-(R)-17-(acetylamino-methyl)-19-methoxy-2,11-dioxo-13-oxa-3,10-diaza-tricyclo[15.3.1.0*4,9*]henicosa-[(21),4(9),5,7,17,19-hexaen-12-yl]-hexanoic acid hydroxyamide, N-hydroxy-6-(R)-(13-methoxy-6,18-dioxo-5,6,7,9,10,11,12,13,18,19-decahydroindolo[2,3-i][4,1,12]benzoxadiazacyclotetradecin-7-yl)hexanamide, N-hydroxy-6-(R)-(13-methoxy-6,18-dioxo-6,7,8,9,10,11,12,13,18,19-decahydro-5H-indolo[2,3-i][1,4,12]benzotriazacyclotetradecin-7-yl)hexanamide, 6-(R)-(13-methoxy-6,18-dioxo-5,6,7,9,10,11,12,13,18,19-decahydroindolo[2,3-i][4,1,12]benzoxadiazacyclotetradecin-7-yl)hexanoic acid, N-hydroxy-6-(R)-(16-methoxy-6,18-dioxo-5,6,7,9,10,11,12,13,18,19-decahydroindolo[2,3-i][4,1,12]benzoxadiazacyclotetradecin-7-yl)hexanamide, 6-(R)-(6,19-dioxo-5,6,7,9,10,11,12,17,18,19-decahydroindolo[2,3-h][4,1,11]benzoxadiazacyclotetradecin-7-yl)-N-hydroxyhexanamide and 6-(R)-(6,20-dioxo-6,7,9,10,11,12,13,18,19,20-decahydro-5H-indolo[2,3-i][4,1,12]benzoxadiazacyclopentadecin-7-yl)-N-hydroxyhexanamide.

4. A compound according to claim 1 selected from the group consisting of:
6-((Z)—(S)-19-methoxy-2,11-dioxo-13-oxa-3,10-diaza-tricyclo[15.3.1.0*4,9*]henicosa-1(21),4,6,8,15,17,19-heptaen-12-yl)-hexanoic hydroxamic acid, 6-((Z)—(R)-19-methoxy-2,11-dioxo-13-oxa-3,10-diaza-tricyclo[15.3.1.0*4,9*]henicosa-1(21),4,6,8,15,17,19-heptaen-12-yl)-hexanoic hydroxamic acid, (S)-2-(6,18-dioxo-5,6,7,9,10,11,12,13,18,19-decahydrobenzo[5,6][1,4,7]-oxadiazacyclo-tetradecino[10,9-b]indol-7-yl)-N-hydroxyacetamide

5. A pharmaceutical composition containing at least one compound according to claim 1 as the active ingredient in mixtures with at least one pharmaceutically acceptable vehicle and/or excipient.

6. (canceled)

7. Method for treating a pathological state for which the modulation of HDAC6 activity would result at improving the health of the patient, said method comprising administering an effective amount of the pharmaceutical composition of claim 5 to a patient in need thereof.

8. Method according to claim 7 where the pathological state is a cancer disease, a neuronal disease, an inflammatory disease or Plasmodium infections.

9. Method according to claim 8 where the cancer disease is cancer of the breasts, pancreas, lung, colon, pleura, peritoneum, face and neck, kidney, bladder, brain, prostate, ovaries or eyes.

10. Method according to claim 9 where the cancer is a metastatic form of cancer.

11. Method according to claim 8 where the inflammatory disease is rheumatoid arthritis.

12. A pharmaceutical composition comprising a compound according to claim 1 together with a pharmaceutically acceptable excipient.

13. A method of treatment of a patient affected by a cancer disease comprising the administration of a compound according to claim 1.

14. Process for synthesizing compounds of claim 1, where X is NHCO, with the nitrogen atom linked to the phenyl moiety, R¹, Y, Z, Ar, m and n being as defined previously, said process comprising reacting compound of formula II 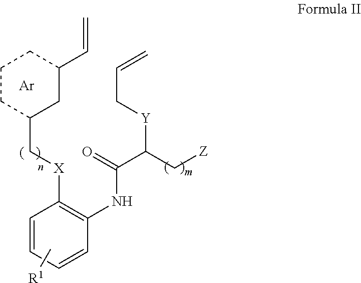
wherein R¹, Y, Z, Ar, m and n are as defined previously and X is NHCO, with the nitrogen atom linked to the phenyl moiety,
with the (1,3-bis-(2,4,6-trimethylphenyl)-2-imidazolidinylidene)dichloro(o-isopropoxyphenylmethylene)ruthenium in an aprotic solvent selected from the group comprising toluene or dichloroethane, at reflux temperature for up to 48 hours.
